(19) United States
(12) Patent Application Publication
(10) Pub. No.: US 2024/0293034 A1
(43) Pub. Date: Sep. 5, 2024

(54) CORONARY DIAGNOSTIC ASSEMBLY WITH GUIDEWIRE FOR BOTH ASSISTING CARDIAC STIMULATION AND MEASURING CARDIAC FLOW RESERVE

(71) Applicant: ELECTRODUCER, Grenoble (FR)

(72) Inventor: Benjamin FAURIE, Grenoble (FR)

(21) Appl. No.: 18/261,727

(22) PCT Filed: Jan. 11, 2022
(86) PCT No.: PCT/EP2022/050369

§ 371 Date: Jul. 17, 2023

(30) Foreign Application Priority Data
Jan. 19, 2021 (FR) 2100508

(51) Int. Cl. A61B 5/0215; A61B 5/00; A61B 17/22

(52) U.S. Cl. CPC A61B 5/0215; A61B 5/6851; A61B 5/6852; A61B 2017/22001

(57) ABSTRACT

The application includes a coronary diagnostic assembly, the wire guide of which serves both to measure transstenotic pressures for a fractional flow reserve (FFR), by means of the pressure sensor integrated into the wire guide, and to perform cardiac pacing during said FFR measurement.

### TECHNICAL FIELD

The present invention relates to a coronary diagnostic assembly, where appropriate for coronary angioplasty.

Coronary angioplasty is also referred to as transluminal dilation, percutaneous transluminal coronary angioplasty (PTCA) or percutaneous coronary intervention (PCI).

The present invention relates more particularly to improving the diagnosis of coronary insufficiency, in particular with a view to coronary angioplasty.

### PRIOR ART

Coronary angioplasty involves treating a narrowed coronary artery, for example in the case of coronary heart disease, by dilating it with the aid of a probe fitted with an inflatable balloon at its end.

The probe mounted on a metal guide is introduced from a (radial or femoral) peripheral artery into the pathological artery. A metal guidewire is used to get past the stenosis or occlusion. It serves as a guide for positioning the angioplasty probe in a stable manner.

In the vast majority of cases, balloon angioplasty is supplemented by the placement of a stent, a small metal prosthesis which is crimped onto the balloon.

Before performing coronary angioplasty, the practitioner performs a diagnosis.

Thus, within the context of the hemodynamic study of coronary stenoses, the use of the coronary physiology is very often necessary and has shown its diagnostic and prognostic benefits. Its widely established use across the world improves the prognosis of patients.

More particularly, measurement of the coronary reserve or fractional flow reserve (FFR) is a simple and recognized method for invasively evaluating the functional impact of a coronary lesion. The FFR principle is based on a measurement of trans-stenotic pressure during maximum vasodilatation (hyperemia), thus making it possible to quantify the impact of a specific coronary lesion. The FFR measurement has become the standard reference index of hemodynamic significance for guiding the revascularization of intermediate coronary lesions.

The FFR is easily measured during coronary angiography by using a pressure guide to calculate the ratio of coronary pressure distal to a stenosis, or a diseased segment, to the aortic pressure under conditions of maximal myocardial hyperemia.

An FFR of 1.0 is widely accepted as normal. An FFR of less than 0.80 is generally considered to be associated with coronary ischemia and to indicate revascularization rather than conservative management.

An FFR can confirm or rule out coronary stenosis (narrowing). A 70% stenosis may not need a stent and, conversely, a 50% stenosis may need one.

The current standard method of measuring the FFR is to insert a pressure guide into the coronary artery, while the hyperemic agent, usually adenosine, is administered by intracoronary bolus.

To date, implementation of a coronary physiology, in particular an FFR, remains complex and requires, as explained above, the administration of drugs to bring about hyperemia, the best known of which drugs is adenosine. Delivered by an intracoronary route, this drug is a vector of undesirable side effects, the most common of which are cardiac arrhythmias such as bradycardia or unpleasant or even dangerous cardiac pauses that generate discomfort of the vagal type or transient cardiac arrests in the patient.

The use of this technique is therefore sometimes limited by the fear of encountering this type of complication or side effect.

There is therefore a need to improve coronary diagnosis, more particularly of coronary physiology by FFR, in particular with a view to angioplasty, in order to overcome the above drawbacks.

The object of the invention is to meet this need at least in part.

### DISCLOSURE OF THE INVENTION

To do this, the subject matter of the invention is, according to a first alternative, a coronary diagnostic assembly, comprising:
an accessory catheter, intended to be introduced into a peripheral artery or vein of the human body;
a sleeve adapted to be engaged around the accessory catheter, the sleeve being made of electrically conductive material over at least part of its outer periphery, such that, when the accessory catheter is introduced into the peripheral artery or vein of the human body, the conductive periphery of the sleeve is in contact with the subcutaneous tissue of the body or with the wall of the artery or of the vein, the sleeve being further connected to an electrical connection, itself connected to an electrode of a cardiac stimulator outside the body;
a guidewire intended to be introduced into the insertion sheath of the accessory catheter, the guidewire comprising, in its distal portion, a pressure sensor for measuring coronary reserve and, in its proximal portion, a metal part further serving as a connection to the other electrode of the external cardiac stimulator and also as a connection to the receiver of the pressure measured by the sensor.

According to an advantageous feature, the electrode of the cardiac stimulator connected to the electrically conductive sleeve engaged around the insertion sheath of the accessory catheter is the anode, while the one connected to the metal part of the guidewire is the cathode.

According to an advantageous variant embodiment, the electrically conductive sleeve is formed as a single piece made of conductive material, for example carbon.

Preferably, the sleeve is formed by a sheath comprising on its outer periphery an electrically conductive coating, for example a coating of carbon.

Advantageously, the sleeve is elastic so as to be able to engage on peripheral arterial or venous catheter sheaths of different diameters, typically external diameters of between 1.6 and 20 mm.

The invention also relates, according to a second alternative, to a coronary diagnostic assembly comprising:
an accessory catheter, intended to be introduced into a peripheral artery or vein of the human body, the accessory catheter comprising at least one tubular insertion sheath and at least one electrically conductive element, of which a distal portion is exposed on at least one part of the outer periphery of the sheath in such a way as to be in contact with the subcutaneous tissue of the body or with the wall of the peripheral artery or vein, and of which a proximal portion, accessible from outside the body (C), comprises an electrical connection so as to serve as a connection to an electrode of a cardiac stimulator outside the body;
a guidewire intended to be introduced into the insertion sheath of the accessory catheter, the guidewire comprising, in its distal portion, a pressure sensor for measuring the coronary reserve and, in its proximal portion, a metal part further serving as a connection to the other electrode of the external cardiac stimulator and also as a connection to the receiver of the pressure measured by the sensor.

The accessory catheter can be an introducer inserted into the radial or femoral artery of a patient.

According to an advantageous feature, the electrode of the cardiac stimulator connected to the accessory catheter is the anode, while the one connected to the metal part of the guidewire is the cathode.

According to an advantageous variant embodiment, the electrically conductive element of the accessory catheter is a wire or a metal band housed at least partially within the thickness of the sheath, of which a distal portion is exposed at the outer periphery of the sheath.

Advantageously, the cross section of the wire or of the metal band being between 0.25 and 5 mm².

The pressure sensor can be a piezoelectric or fiber-optic sensor which is connected to the central pressure measurement unit respectively by an electric wire or a fiber optic. The inventor has overcome a prejudice on this point, because generating an electric current for the desired cardiac stimulation is added to the electrical or fiber-optic signal for the pressure measurement. It also turns out that no disturbance is induced, i.e. the pressure measurement is not disturbed by the electrical signal of the cardiac stimulation.

The assembly according to the invention can additionally constitute a coronary angioplasty assembly with placement of a stent, the guidewire being adapted for advancing the stent.

The invention also relates to a method of diagnosis and, where appropriate, of cardiac intervention, comprising the following steps:
i/ engaging a sleeve around an accessory catheter, the sleeve being made of electrically conductive material over at least part of its outer periphery and further comprising an electrical connection to an electrode of a cardiac stimulator outside the body;
ii/ introducing an accessory catheter into a peripheral artery or vein of a human body, such that the conductive periphery of the sleeve is in contact with the subcutaneous tissue of the body or with the wall of the artery or the vein;
iii/ introducing a guidewire into the insertion sheath of the accessory catheter, the guidewire comprising at its distal end a pressure sensor and, at its proximal part, a metal part further serving as a connection to the other electrode of the external cardiac stimulator;
iv/ measuring the pressure with the aid of the sensor as a measurement of the coronary reserve and, simultaneously to the measurement of the coronary reserve, direct cardiac stimulation on the guidewire by means of the external cardiac stimulator;
v/ if applicable, in the event of placement of a stent, cardiac stimulation by means of the external cardiac stimulator, the guidewire being held in place once step
iv/ has been carried out.

The invention relates finally to a method of diagnosis and, where appropriate, of cardiac intervention, comprising the following steps:
i′/ introducing an accessory catheter into a peripheral artery or vein of a human body, the accessory catheter comprising at at least one tubular insertion sheath and at least one electrically conductive element, a distal portion of which is exposed over at least a part of the outer periphery of the sheath so as to be in contact with the subcutaneous tissue of the body or with the wall of the peripheral artery or vein, and of which a proximal portion, accessible from outside the body (C), comprises an electrical connection, so as to serve as a connection to an electrode of a cardiac stimulator outside the body;
ii′/ introducing a guidewire into the insertion sheath of the accessory catheter, the guidewire comprising, at its distal end, a pressure sensor and, at its proximal end, a metal part further serving as a connection to the other electrode of the external cardiac stimulator;
iii′/ measuring the pressure with the aid of the sensor as a measurement of the coronary reserve and, simultaneously to the measurement of the coronary reserve, direct cardiac stimulation on the guidewire by means of the external cardiac stimulator;
iv′/ if applicable, in the event of placement of the stent, cardiac stimulation by means of the external cardiac stimulator, the guidewire being held in place once step iii′/ has been carried out.

Thus, the invention essentially consists of a coronary diagnostic assembly whose guidewire serves both to measure trans-stenotic pressures for a coronary reserve (FFR), by the pressure sensor integrated in the guidewire, and to carry out the cardiac stimulation during this FFR measurement.

By inducing direct cardiac stimulation on a guidewire intended for FFR measurement, the assembly according to the invention makes it possible to avoid undesirable side effects linked to cardiac arrhythmias or conduction disorders such as bradycardia or a harmful cardiac pause, and hyperemia that has to be carried out before an FFR measurement.

Thus, the invention makes the FFR measurement procedure safer for the practitioner and the patient and makes it possible to carry out this type of physiological study in any type of patient, even in the event of a theoretical contraindication to the use of adenosine, which is necessary for carrying out hyperemia. An example of a contraindication is a first-degree atrioventricular block.

In addition, the stimulation intensity required for cardiac shutdown during FFR measurement is low. Typically, the intensity of the current delivered, with a view to cardiac shutdown, can range from 4 to 25 mA and the voltage delivered can range from 1 to 15 volt.

This not only guarantees that the patient is protected from undesirable side effects (bradycardia, cardiac pause), but also that the patient feels little or no sensation related to the cardiac stimulation, given that the endovascular electrode, typically the anode, is of large dimensions.

The surgeon or surgeons in charge of the operation can thus easily connect the electrode, typically the anode of the cardiac stimulator, to the conductive sleeve engaged around the accessory catheter, then as usual connect the other electrode, typically the cathode, to the guidewire of the catheter/stent assembly or the balloon alone. This is important especially for weaker patients.

In summary, the advantages of a coronary diagnostic assembly are many, among which the following may be mentioned:
the coronary FFR measurement procedure is made safer by eliminating all the undesirable side effects linked to ingestion of drugs, such as the adenosine that is needed to effect hyperemia prior to the FFR;
a guarantee of temporary stimulation with a view to carrying out “emergency” stimulation, that is to say in an emergency situation, which is desired because of the lower impedance of the vascular system encountered by the electrical stimulation current, since the sleeve around an accessory venous catheter or the latter, when it incorporates the metal part to be connected to the electrode, is directly in contact with said system;
the possibility of implementation on any existing peripheral venous or arterial catheter, since a conductive sleeve simply has to be engaged around a peripheral venous or arterial catheter, before its introduction into a peripheral vein or artery and an electrical connection to the guidewire inserted into the catheter has to be performed.

The only relative constraint of the sleeve is at the start of the intervention, during the preparation, and consists in engagement of the sleeve around the insertion sheath of the accessory (peripheral arterial) catheter.

However, this operation is very simple and easy to implement and can be carried out by an assistant or nurse who does not require special skills for this task.

Other advantages and features will become clear on reading the detailed description, which is given by way of a non-limiting example, with reference to the appended figures.

### BRIEF DESCRIPTION OF THE DRAWINGS

FIG. 1 is a schematic view illustrating the use of a diagnostic assembly according to the invention with a peripheral arterial catheter and a stimulation sleeve according to the invention, the catheter being introduced directly into a peripheral artery of a patient.

FIG. 2 shows the arrangement of the guidewire of the assembly according to FIG. 1 within a coronary artery that is to be diagnosed.

FIG. 3 is a perspective view of an electrically conductive sleeve according to the invention intended to be engaged around a peripheral arterial catheter as shown in FIGS. 1 and 2;

FIG. 4 is a partial longitudinal sectional view showing a sleeve according to FIG. 3 engaged around a peripheral arterial catheter.

### DETAILED DESCRIPTION

In the following description, and throughout the present application, the terms “distal” and “proximal” are used with reference to the body of a patient undergoing coronary diagnosis, if necessary followed by coronary angioplasty. Thus, the distal end of a guidewire is the end located farthest inside the patient during the diagnosis.

It should be noted that the various elements are not necessarily shown to scale.

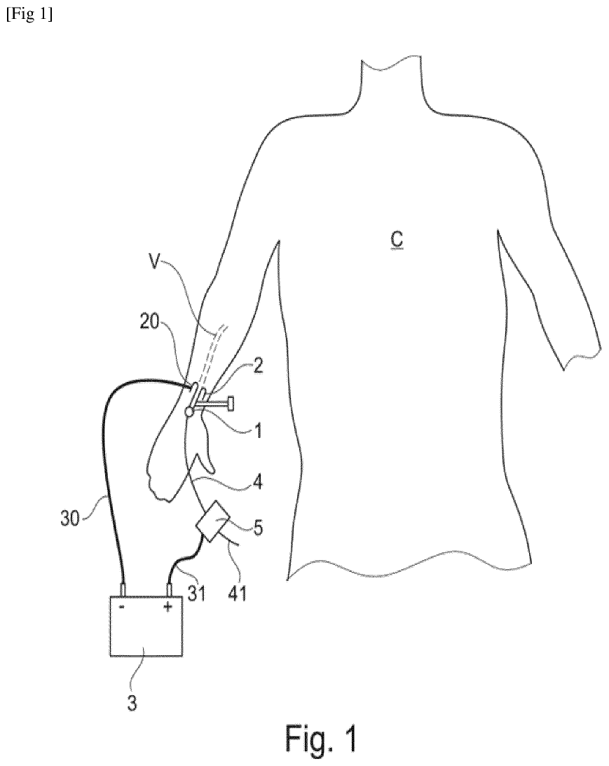

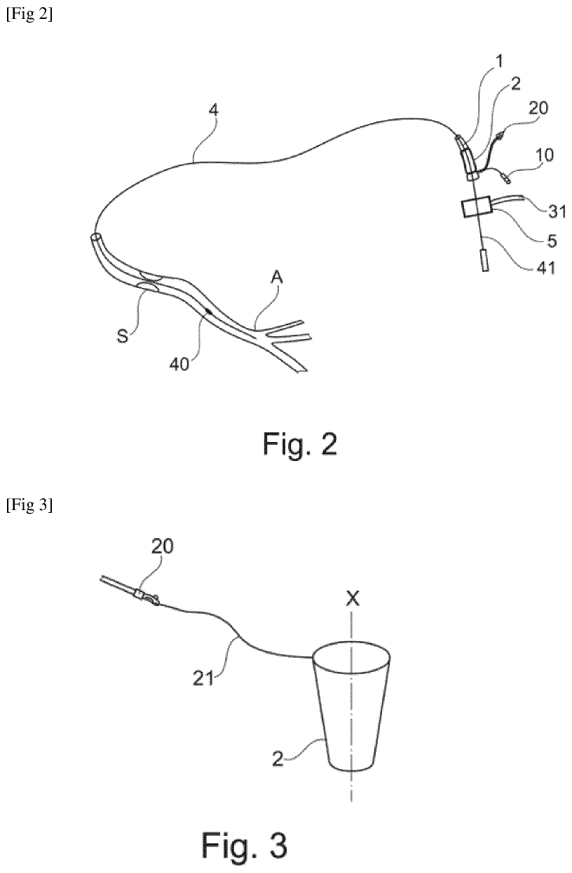

FIGS. 1 and 2 show a coronary diagnostic assembly according to the invention.

A peripheral arterial catheter 1, also called an accessory catheter, is introduced into a peripheral artery V.

Such an arterial catheter 1 can be of small diameter, typically 2 mm or less, and of short length. It meets the standards applying to existing peripheral intravascular catheters. The catheter 1 can comprise an integrated rinsing device 10 with taps, commonly called a “flush”, in order to rinse the inside of the catheter 1 by means of a suitable rinsing liquid.

An electrically conductive sleeve 2 is engaged directly around the peripheral arterial catheter 1. The sleeve 2 is connected to an electrical connection 20, itself connected to an electrode, typically the anode of a cardiac stimulator 3, external to the body C, by way of an electrical power supply wire 30.

A guidewire 4 is introduced into the insertion sheath of the accessory catheter 1.

As is shown in FIG. 2, the guidewire 4 comprises, in its distal portion, a pressure sensor 40 for measuring trans-stenotic pressures in the coronary artery A and, in its proximal portion, a metal part 41 further serving as a connection to the other electrode, typically the cathode of the external cardiac stimulator. To ensure the electrical connection, a clip 5 of the crocodile type is fixed by clipping onto the metal part 41 of the guidewire 4. This clip 5 is connected to the anode of the cardiac stimulator by way of an electrical power supply wire 31.

A sleeve 2 according to the invention is shown in FIG. 3: it is in the form of a tube, of which at least part of the outer periphery is electrically conductive.

This conductive part is connected to an electrical connection 20, typically in the form of a clip, by way of an electrical power supply wire 21. The sleeve 20, the clip 20 and the electrical power supply wire 21 can advantageously constitute a kit ready to be engaged around a catheter 1 and connected to a cardiac stimulator electrode 3.

The sleeve 2 can be of cylindrical or frustoconical shape. Its shape matches as closely as possible the outer shape of the insertion sheath of a peripheral arterial catheter 1.

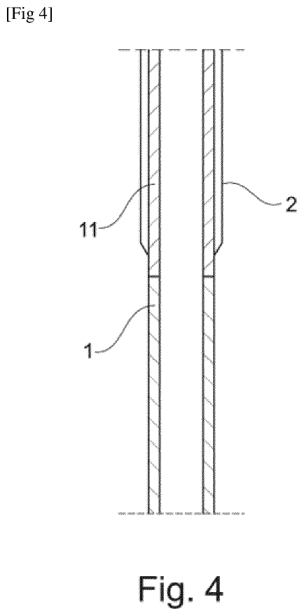

As can be seen in FIG. 4, the thickness of this sleeve 2, typically of the order of a millimeter or less, adds only a slight extra thickness to the sheath 11 of the arterial catheter 1 and therefore does not impede the progress of the latter upon its introduction into a peripheral artery V.

The sleeve 2 can be made in the form of a fully conductive one-piece component or in the form of a component coated with an electrically conductive coating. Carbon can advantageously be chosen as the electrically conductive material.

According to an advantageous variant, the sleeve 2 can be designed such that it is elastic and is thus able to conform to any size of existing peripheral arterial catheter.

In practice, a surgeon or interventionalist seeking to perform a diagnosis jointly with cardiac stimulation, as explained below, begins by positioning the conductive sleeve 2 around a peripheral arterial catheter 1. The placement of the conductive sleeve being very simple and easy, it can be done by an assistant or nurse without having to use a particular technique.

The placement is achieved once the sleeve 2 has been engaged and arranged around the insertion sheath of the arterial catheter 1 such that said sleeve 2 touches either a subcutaneous region of the patient or the wall of the peripheral vein of the patient.

Once this placement has been carried out, the electrical connection 20 can be connected directly to the anode of an external cardiac stimulator 3 by way of a connection wire 30.

Usually, a clip 5, such as a crocodile clip, can in turn be fixed by clipping onto the metal part 41 of the guidewire 4 which is inserted in the sheath 11 of the peripheral catheter 1 and whose pressure sensor 40 is positioned in the coronary artery that is to be diagnosed. This clip 5 is connected to the cathode of the external cardiac stimulator 3 by way of a connection wire 31.

Thus, the temporary cardiac stimulation for achieving the desired cardiac shutdown during a measurement of trans-stenotic pressure can take place between the cathode electrically connected to the guidewire 4 and the anode electrically connected to the sleeve 2 according to the invention around the peripheral venous catheter 1.

The method of coronary diagnosis, and if necessary of cardiac intervention, implemented by the above-described assembly is now described.

This method is applied when a practitioner wishes to perform a coronary diagnosis, in particular a measurement of the coronary reserve, because he or she suspects a stenosis in a coronary artery A, such as the stenosis designated S in FIG. 2.

Step i/: a nurse or a practitioner proceeds to engage the electrically conductive sleeve 2 around a peripheral (accessory) catheter 1.

Step ii/: the practitioner then introduces the catheter 1 into a peripheral artery or vein of a human body C, such that the conductive periphery of the sleeve 2 is in contact with the subcutaneous tissue of the body or with the wall of the artery or vein.

Step iii/: the practitioner then introduces the guidewire 4 into the insertion sheath 11 of the accessory catheter 1, until it is ensured that the pressure sensor 40 is correctly positioned in the coronary artery A that is to be diagnosed.

He or she can then proceed with the electrical connection to the external cardiac stimulator 3.

Thus, the nurse or the practitioner connects on the one hand the sleeve 2 to an electrode, typically the anode of the external cardiac stimulator 3, and on the other hand the metal part 40 of the guidewire 4 to the other electrode of the stimulator 3.

Step iv/: after bringing about hyperemia in the patient, the practitioner can measure the trans-stenotic pressure with the aid of the sensor 40, as a measure of the coronary reserve.

Simultaneously with the measurement of the coronary reserve or FFR, direct cardiac stimulation is performed on the guidewire 4.

More precisely, the pressure or FFR is measured once the practitioner is sure that the adenosine administered to the patient has had its effect.

The cardiac stimulation can be triggered before the initiation of the pressure measurement, typically before or at the same time as the practitioner ensures that the adenosine has taken effect. The practitioner can also decide to administer adenosine under cardiac stimulation by means of the guidewire according to the invention.

As regards the rate of cardiac stimulation, the practitioner can choose from the following two modes:
a stimulation mode that can be described as “sentinel”: the external cardiac stimulation is set to a rhythm threshold lower than that of the patient's heart at rest. As soon as the patient's heartbeat is lower than the threshold, the stimulator is put into operation and the cardiac stimulation is effective via the electrode of the guidewire and the electrode integrated in the sleeve or in the accessory catheter. For example, a patient's heart can beat at 80 beats per minute at rest, and as soon as it beats at 60 beats during the FFR diagnosis, the external cardiac stimulator is triggered;
a permanent stimulation mode according to which the external cardiac stimulator is triggered permanently during the FFR diagnosis and, if necessary, once adenosine is administered, at a frequency higher than the patient's heart rate at rest. For example, if the patient's resting heart rate is 60 beats per minute, the external cardiac stimulator operates continuously at a rate of 80 beats per minute.

Whichever the mode chosen by the practitioner, this rate of stimulation can be easily implemented knowing that, according to the FFR procedure, the heart rate of the patient is monitored initially and then continuously throughout the intervention (diagnosis possibly followed by coronary angioplasty).

By means of cardiac stimulation thus being carried out simultaneously, the patient avoids all the risks associated with bradycardia or a harmful cardiac pause that could be caused by hyperemia.

The coronary diagnostic assembly that has just been described can also be used to perform coronary angioplasty.

In fact, at the end of step iv/, if an identified stenosis S requires the placement of a stent in the coronary artery A, then, having chosen a guidewire 4 which also incorporates an inflatable balloon with stent, it is possible to put the stent in place without having removed any of the components of the assembly.

Thus, according to a step v/, during the deployment of the balloon and of the stent, the practitioner performs another cardiac stimulation by means of the external cardiac stimulator, the guidewire being held in place once step iv/ has been performed.

The invention is not limited to the examples that have just been described; it is possible in particular to combine together features of the examples illustrated within variants not illustrated.

Other variants and improvements may be provided without departing from the scope of the invention.

If, in all of the examples illustrated, the guidewire integrating both a pressure sensor and a metal part as an external cardiac stimulation electrode is used in a coronary diagnostic assembly, it can be used for taking the trans-stenotic pressure in cardiac cavities and for cardiac stimulation, as per the teaching of the patent applications FR3079404, EP3522800, EP3280338 (B1), FR3057153 and WO2019/185880, in the context of implantation of an aortic valve by transcatheter aortic valve implantation (TAVI).

A guidewire for a coronary diagnostic (FFR) assembly can have a small diameter, typically equal to 0.035 mm (0.014 inch), while a guidewire for an assembly measuring pressure within cardiac cavities can be of larger diameter, typically of the order of 0.9 mm (0.35 inch).

1. A coronary diagnostic assembly, comprising:
an accessory catheter, intended to be introduced into a peripheral artery or vein of the human body;
a sleeve adapted to be engaged around the accessory catheter, the sleeve being made of electrically conductive material over at least part of its outer periphery, such that, when the accessory catheter is introduced into the peripheral artery or vein of the human body, the conductive periphery of the sleeve is in contact with the subcutaneous tissue of the body or with the wall of the artery or of the vein, the sleeve being additionally connected to an electrical connection, itself connected to an electrode of a cardiac stimulator outside the body;
a guidewire, intended to be introduced into the insertion sheath of the accessory catheter, the guidewire comprising, in its distal portion, a pressure sensor for measuring coronary reserve and, in its proximal portion, a metal part further serving as a connection to the other electrode of the external cardiac stimulator and also as a connection to the receiver of the pressure measured by the sensor.

2. The assembly as claimed in claim 1, wherein the electrode of the cardiac stimulator connected to the electrically conductive sleeve engaged around the insertion sheath of the accessory catheter is the anode, while the one connected to the metal part of the guidewire is the cathode.

3. The assembly as claimed in claim 1, in which the electrically conductive sleeve is formed as a single piece made of conductive material.

4. The assembly as claimed in claim 3, wherein the sleeve is formed by a sheath comprising on its outer periphery an electrically conductive coating.

5. The assembly as claimed in claim 3, wherein the sleeve is elastic so as to be able to engage on peripheral arterial or venous catheter sheaths of different diameters, typically external diameters of between 1.6 and 20 mm.

6. A coronary diagnostic assembly, comprising:
an accessory catheter, intended to be introduced into a peripheral artery or vein of the human body, the accessory catheter comprising at least one tubular insertion sheath and at least one electrically conductive element, of which a distal portion is exposed on at least one part of the outer periphery of the sheath in such a way as to be in contact with the subcutaneous tissue of the body or with the wall of the peripheral artery or vein, and of which a proximal portion, accessible from outside the body (C), comprises an electrical connection so as to serve as a connection to an electrode of a cardiac stimulator outside the body;
a guidewire intended to be introduced into the insertion sheath of the accessory catheter, the guidewire comprising, in its distal portion, a pressure sensor for measuring the coronary reserve and, in its proximal portion, a metal part further serving as a connection to the other electrode of the external cardiac stimulator and also as a connection to the receiver of the pressure measured by the sensor.

7. The assembly as claimed in claim 6, wherein the electrode of the cardiac stimulator connected to the accessory catheter is the anode, while the one connected to the metal part of the guidewire is the cathode.

8. The assembly as claimed in claim 6, in which the electrically conductive element of the accessory catheter is a wire or a metal band housed at least partially within the thickness of the sheath, of which a distal portion is exposed at the outer periphery of the sheath.

9. The assembly as claimed in claim 8, wherein the cross section of the wire or of the metal band is between 0.25 and 5 mm².

10. The assembly as claimed in claim 1, wherein the pressure sensor is a piezoelectric or fiber-optic sensor.

11. The assembly as claimed in claim 1, additionally comprising a coronary angioplasty assembly with placement of a stent, the guidewire being adapted for advancing the stent.
